(19) United States
(12) Patent Application Publication
(10) Pub. No.: US 2014/0277306 A1
(43) Pub. Date: Sep. 18, 2014

(54) Method and Apparatus Pertaining to Free-Standing Wireless Temperature Sensors

(71) Applicant: MEDLINE INDUSTRIES, INC., Mundelein, IL (US)

(72) Inventor: Andrew Giles, Libertyville, IL (US)

(73) Assignee: MEDLINE INDUSTRIES, INC., Mundelein, IL (US)

(21) Appl. No.: 13/832,594

(22) Filed: Mar. 15, 2013

(51) Int. Cl. A61F 7/00

(52) U.S. Cl. CPC A61F 7/0097
USPC 607/107

(57) ABSTRACT

A control circuit wirelessly couples to at least one free-standing wireless temperature sensor that the control circuit employs to monitor a temperature corresponding to a patient and thereby provide monitored-temperature information that the control circuit then uses to control personal warming of the patient. By one approach the control circuit can also wirelessly couple to one or more non-free-standing wireless temperature sensors the control circuit can also use to provide additional monitored-temperature information regarding the patient, which additional monitored-temperature information the control circuit can further use when controlling personal warming of the patient.

### TECHNICAL FIELD

This invention relates generally to patient warming.

### BACKGROUND

Personal patient-warming apparatuses are known in the art. Being “personal,” these apparatuses do not serve in any meaningful way to warm a general area (such as a room). Instead, these apparatuses serve to provide local-to-a-patient warming for the benefit of an individual patient (typically during the administration of a medical-services procedure such as but not limited to an operation). While some of the delivered warmth will typically escape beyond the patient themselves, the focus of the warmth delivery mechanism is intended and designed to primarily warm the patient as versus the local environment. While sometimes employed to improve the patient's comfort, patient warming can also provide important patient treatment and/or recovery support as well.

One approach to locally warming a patient relies upon electric heating elements that convert electricity into heat. In some cases the patient lies atop a patient-support surface that includes one or more such electric heating elements. By another approach electric heating elements serve to heat air that is delivered in close proximity to the patient (via, for example, a warm-air blanket).

In some cases the delivery of warmth to the patient includes using sensed-temperature feedback to modulate and control, to some extent, the temperature of the warming medium. When delivering warmed air to a warm-air blanket, for example, the heater/blower assembly may include a temperature sensor that connects via an electrical conductor to an integral temperature control circuit. As another example, a patient-support surface that includes an integral electric heating element may have one or two temperature sensors disposed therein that connect to a corresponding control circuit via, for example, an optical fiber.

These solutions are often useful and generally serve as intended. The applicants have determined, however, that there can be application settings where such solutions do not always provide a desired degree of relevant control and/or convenience of use.

### BRIEF DESCRIPTION OF THE DRAWINGS

The above needs are at least partially met through provision of the method and apparatus pertaining to free-standing wireless temperature sensors described in the following detailed description, particularly when studied in conjunction with the drawings, wherein:

FIG. 1 comprises a flow diagram as configured in accordance with various embodiments of the invention;

FIG. 2 comprises a block diagram as configured in accordance with various embodiments of the invention;

FIG. 3 comprises a perspective schematic view as configured in accordance with various embodiments of the invention;

FIG. 4 comprises a perspective view as configured in accordance with various embodiments of the invention;

FIG. 5 comprises a perspective schematic view as configured in accordance with various embodiments of the invention; and

FIG. 6 comprises a perspective schematic view as configured in accordance with various embodiments of the invention.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these various embodiments, a control circuit wirelessly couples to at least one free-standing wireless temperature sensor that the control circuit employs to monitor a temperature corresponding to a patient and thereby provide monitored-temperature information that the control circuit then uses to control personal warming of the patient. By one approach the control circuit can also wirelessly couple to one or more non-free-standing wireless temperature sensors the control circuit can also use to provide additional monitored-temperature information regarding the patient, which additional monitored-temperature information the control circuit can further use when controlling personal warming of the patient.

These teachings are highly flexible in practice. The aforementioned free-standing wireless temperature sensor can be temporarily installed, for example, on the patient, underneath the patient, or even within the patient as desired. The aforementioned non-free-standing wireless temperature sensor, in turn, can be non-temporarily installed in (or on, as appropriate), for example, a patient support surface, a pneumatic pathway that conveys warmed air to the patient, or a warm-air blanket, to note but a few examples in these regards.

Those skilled in the art will also appreciate the scalability of these teachings. These approaches will readily accommodate, for example, any number and combination of free-standing wireless temperature sensors, non-free-standing wireless temperature sensors, and even non-wireless temperature sensors.

So configured, such a free-standing wireless temperature sensor can be readily and easily deployed in a given application setting to best accommodate the circumstances presented by a given patient having certain warming needs. This flexibility permits the warming technician to craft a custom configuration that is better suited to a given patient/circumstance than can be expected with typical fixed-configuration temperature sensors. As but one example in these regards, warmth being provided to pediatric patients, notwithstanding the diminutive size of the patient, can be readily and effectively monitored and controlled.

These and other benefits may become clearer upon making a thorough review and study of the following detailed description. Referring now to the drawings, and in particular to FIG. 1, an illustrative process 100 that is compatible with many of these teachings will now be presented.

Referring momentarily to FIG. 2, this process 100 is presumably carried out by a corresponding apparatus 200 and in particular by a control circuit 201 of choice as comprises a part of that apparatus 200. Such a control circuit 201 can comprise a fixed-purpose hard-wired platform or can comprise a partially or wholly programmable platform. These architectural options are well known and understood in the art and require no further description here. This control circuit 201 is configured (for example, by using corresponding programming as will be well understood by those skilled in the art) to carry out one or more of the steps, actions, and/or functions described herein.

By one approach the control circuit 201 operably couples to an optional memory 202. This memory 202 may be integral to the control circuit 201 or can be physically discrete (in whole or in part) from the control circuit 201 as desired. If desired, this memory 202 can also be local with respect to the control circuit 201 (where, for example, both share a common circuit board, chassis, power supply, and/or housing).

This memory 202 can serve, for example, to non-transitorily store the computer instructions that, when executed by the control circuit 201, cause the control circuit 201 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as an erasable programmable read-only memory (EPROM).)

The control circuit 201 operably couples to a heating mechanism 203 of choice and the control circuit 201 is configured to control, to at least some extent, the heat output of the heating mechanism 203. The present teachings will accommodate a variety of control strategies in these regards. By one approach, for example, the control circuit 201 can switch the heating mechanism 203 between on and off states. By another approach, the control circuit 201 can cause the heating mechanism 203 to make selective use of a variety of heat-output levels. As will be made clearer below, the control circuit's use of this heating mechanism 203 can be informed by temperature feedback information provided by one or more temperature sensors.

This apparatus 200 also includes a wireless receiver 204 that also operably couples to the control circuit 201. This wireless receiver 204 is configured to compatibly receive wireless communications from wireless temperature sensors described further herein. If desired, this wireless receiver 204 can comprise a wireless transceiver to thereby permit the control circuit 201 to transmit information as well as receive information. Transmitted information might include, by way of example, polling instructions to wireless temperature sensors to cause the latter to report their presently-sensed temperatures and/or to provide a batch report of a plurality of previously-sensed temperatures.

By one approach, and by way of an example without intending any limitations in these regards, this wireless receiver 204 can comprise a Bluetooth-compatible wireless receiver. Wireless receivers in general comprise a very well understood area of prior art endeavor. Accordingly, for the sake of brevity further elaboration in these regards is not provided here.

Per these teachings this apparatus 200 also includes at least one free-standing wireless temperature sensor 205. Electronic temperature sensors are very well known in the art and the present teachings will accommodate any of a variety of approaches in these regards. As the present teachings are not sensitive to the selection of any particular approach in these regards, further details regarding such sensors is not provided here.

Being “wireless,” the free-standing wireless temperature sensor 205 includes at least one wireless transmitter of choice. When the wireless receiver 204 as corresponds to the control circuit 201 comprises a Bluetooth-compatible receiver, for example, this wireless transmitter can, in turn, comprise a Bluetooth-compatible transmitter.

That said, there are numerous choices as regards the wireless link between this wireless temperature sensor 205 and the control circuit's wireless receiver 204. As but one additional example in these regards, the wireless transmitter can comprise a passive radio-frequency identification (RFID) tag and the wireless receiver 204 can comprise an RFID-tag reader. Such an approach can serve well when the application setting requires that the free-standing wireless temperature sensor 205 have an extremely small size and form factor.

As used herein, the expression “free-standing” will be understood to refer to a fielded configuration that is not intended or designed for a static installation configuration as an integral part of some larger apparatus, component, or assembly. FIG. 3 offers some illustrative example in these regards. In this figure a patient 300 lies atop a patient support surface 301. As a first illustrative example a first free-standing wireless temperature sensor 205-1 that includes a small adhesive patch 302 is placed on the patient's torso in a useful location of convenience. As a second illustrative example a second free-standing wireless temperature sensor 205-2 is loosely disposed (again, at a useful/convenient location) between the patient's back and the patient support surface 301. And as yet another illustrative example a third free-standing wireless temperature sensor 205-3 is attached to an esophageal probe 303 and placed within the patient 300 to assess the patient's interior temperature. In all of these examples the temperature sensor is positionable at the will of the technician and hence is “free-standing.”

Though “free-standing,” these teachings will accommodate using, or not using, attachment mechanisms to temporarily fix the free-standing wireless temperature sensor 205 at a desired location. Example attachment mechanisms include but are not limited to an adhesive (as noted above), a clip, clasp, pin, or the like, magnets, and so forth. “Free-standing” sensors can also include temporarily-implantable sensors (including both tethered and untethered sensors configured to be temporarily placed within a living body).

Returning again to FIG. 2, in addition to one or more free-standing wireless temperature sensors 205 the apparatus 200 can also include, if desired, one or more non-free-standing wireless temperature sensors 206 that also operably couple to the control circuit 201 via the wireless receiver 204. Being a “non-free-standing” wireless temperature sensor 206, these temperature sensors 206 typically comprise an integral part of another apparatus.

As one example in these regards, FIG. 4 illustrates a patient support surface 301 comprising a pad having, in this case, two non-free-standing wireless temperature sensors 206-1 and 206-2 disposed within the pad very proximal to the patient-side of the pad (for example, within one centimeter of the pad's upper surface and hence the patient). As another example, and referring to FIG. 5, a non-free-standing wireless temperature sensor 206-3 is disposed within, and is a part of, the heated-air output port of a heating component 203 that includes a blower 501 and an electric heating element 502. And as yet another example, and referring now to FIG. 6, a non-free-standing wireless temperature sensor 206-4 is disposed within a warm-air blanket 601 that is configured to receive warmed air via a conduit 602 and to release that warmed air over a patient via a plurality of small orifices 603 formed on the underside thereof.

It will be understood that the various foregoing examples of free-standing wireless temperature sensors and non-free-standing wireless temperature sensors are offered for the purpose of illustration and are not intended to serve as an exhaustive listing in these regards. In fact, there are numerous other configurations by which a given wireless temperature sensor can serve in either a free-standing or a non-free-standing manner.

Referring again to FIG. 2, if desired, by one optional approach the apparatus 200 can also include one or more temperature sensors 207 that are not wireless but which operably couple to the control circuit 201 via some non-wireless mechanism such as one or more electrical conductors, optical fibers, or the like. Such a non-wireless temperature sensor 207 can be free-standing or non-free-standing as desired. In such a case, the aforementioned free-standing wireless temperature sensors 205 can serve to supplement and/or compliment the information provided by the non-wireless temperature sensors 207.

Referring again to FIG. 1, such a control circuit 201, at 101, monitors a temperature corresponding to a patient via the at least one free-standing wireless temperature sensor 205 to thereby provide monitored-temperature information. This monitoring can occur, if desired, as a series of sensed-temperature samples. The sampling rate can vary as desired with examples including one sample per 0.1 second, one sample per 1.0 second, one sample per 5.0 seconds, and so forth (with more-frequent and less-frequent sampling rates certainly being possible).

When the control circuit 201 has access to at least one non-free-standing wireless temperature sensor 206, at 102 this process 100 can also provide for the control circuit 201 monitoring a temperature corresponding to the patient via that at least one non-free-standing wireless temperature sensor 206 to thereby provide additional monitored-temperature information (“additional” in view of the monitored-temperature information being provided by the free-standing wireless temperature sensor(s) 205). As with the free-standing wireless temperature sensor 205 this non-free-standing wireless temperature sensor 206 can be sampled using a desired sampling rate.

At 103 the control circuit 201 then uses the foregoing monitored-temperature information (and the additional monitored-temperature information, when available) to control the personal warming of a patient. As one simple example in these regards the monitored-temperature information serves as real-time feedback as to the present state of that warming activity. Because the free-standing wireless temperature sensor 205 can be placed essentially anywhere in many cases, the technician can place one or more such sensors in locations where the quality and relevance of that feedback information is higher than one skilled in the art expects in such an application setting.

This improved quality of information, in turn, can lead to more effective patient warming (i.e., neither too hot nor too cool) with corresponding salutary benefits for the patient. It will be further appreciated that such benefits attain notwithstanding great variability with respect to patient sizes, shapes, and circumstances as the wireless, free-standing nature of at least some of the temperature sensors permits a technician to fabricate, on the fly, a custom fielding of such a temperature sensor in a way that best suits the immediate need.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

What is claimed is:

1. A method comprising:
by a control circuit that wirelessly couples to at least one free-standing wireless temperature sensor:
monitoring a temperature corresponding to a patient via the at least one free-standing wireless temperature sensor to provide monitored-temperature information;
using the monitored-temperature information to control personal warming of the patient.

2. The method of claim 1 wherein the at least one free-standing wireless temperature sensor is installed on the patient.

3. The method of claim 1 wherein the at least one free-standing wireless temperature sensor is installed within one centimeter of the patient.

4. The method of claim 3 wherein the at least one free-standing wireless temperature sensor is installed underneath the patient.

5. The method of claim 1 wherein the at least one free-standing wireless temperature sensor is installed within a warm-air blanket.

6. The method of claim 1 wherein the control circuit is further operably coupled to at least one non-free-standing wireless temperature sensor, the method further comprising:
monitoring a temperature corresponding to the patient via the at least one non-free-standing wireless temperature sensor to provide additional monitored-temperature information;
using both the monitored-temperature information and the additional monitored-temperature information to control personal warming of the patient.

7. The method of claim 6 wherein the non-free-standing wireless temperature sensor is installed in at least one of:
a patient support surface;
a pneumatic pathway that conveys warmed air to the patient.

8. The method of claim 1 wherein the control circuit wirelessly couples to at least two free-standing wireless temperature sensors and wherein monitoring a temperature corresponding to a patient via the at least one free-standing wireless temperature sensor to provide monitored-temperature information comprises monitoring temperatures corresponding to the patient via both of the at least two free-standing wireless temperature sensors to provide the monitored-temperature information.

9. An apparatus comprising:
at least one free-standing wireless temperature sensor;
a control circuit operably coupled to the at least one free-standing wireless temperature sensor and configured to:
monitor a temperature corresponding to a patient via the at least one free-standing wireless temperature sensor to provide monitored-temperature information;
use the monitored-temperature information to control personal warming of the patient.

10. The apparatus of claim 9 wherein the at least one free-standing wireless temperature sensor is installed on the patient.

11. The apparatus of claim 9 wherein the at least one free-standing wireless temperature sensor is installed within one centimeter of the patient.

12. The apparatus of claim 11 wherein the at least one free-standing wireless temperature sensor is installed underneath the patient.

13. The apparatus of claim 9 wherein the at least one free-standing wireless temperature sensor is installed within a warm-air blanket.

14. The apparatus of claim 9 further comprising:
at least one non-free-standing wireless temperature sensor that operably couples to the control circuit;
and wherein the control circuit is further configured to:
monitor a temperature corresponding to the patient via the at least one non-free-standing wireless temperature sensor to provide additional monitored-temperature information;
use both the monitored-temperature information and the additional monitored-temperature information to control personal warming of the patient.

15. The apparatus of claim 14 wherein the non-free-standing wireless temperature sensor is installed in at least one of:
a patient support surface;
a pneumatic pathway that conveys warmed air to the patient.

16. The apparatus of claim 9 further comprising:
at least a second free-standing wireless temperature sensor;
and wherein the control circuit is configured to monitor temperatures corresponding to the patient via both of the at least two free-standing wireless temperature sensors to provide the monitored-temperature information.
